(19) United States
(12) Patent Application Publication
(10) Pub. No.: US 2009/0292256 A1
(43) Pub. Date: Nov. 26, 2009

(54) Nasogastric tube fastener

(76) Inventor: Ed Cubberly, Frenchtown, NJ (US)

Correspondence Address: B2 PATENT SERVICES, 406 ROSEMONT-RINGOES ROAD, STOCKTON, NJ, 08559, US

(21) Appl. No.: 12/154,335

(22) Filed: May 22, 2008

(51) Int. Cl. A61M 5/32

(52) U.S. Cl. 604/180

(57) ABSTRACT

This invention provides a device for fastening a nasogastric tube in place by attachment to the surface of the patient's nose. The device provides the wearer with a securely positioned tube, not easily moved, but yet comfortable to wear. In addition men with facial hair may be spared the need to shave the upper lip in order to tape the tube in place. The device, being crafted out of a flexible film or fabric provides some degrees of motion for even greater comfort and ease of medical care.

### FIELD OF INVENTION

This invention relates to a self sticking device for securely and comfortably holding a nasogastric tube or similar feeding tube in a patient's nose. Nasogastric tubes are used to feed patients by pumping a solution of glucose, saline or other nutrients directly into the stomach, or they can be used to empty the contents of the stomach by applying a suction or vacuum. In either use, once the nasogastric tube is inserted and placed in the proper position and depth, it must be held in the exact position in order to insure proper function.

### BACKGROUND OF INVENTION

The present invention relates to a device for securely attaching a nasogastric tube to the nose of a patient. Many devices have been described in the currently literature to hold such a tube, which attach to the patients upper lip or to the head using a head gear. These devices can be uncomfortable to wear for extended times and can have limited grip on the tube resulting in slippage and even accidental dislodging of the feeding tube. There still exists a need for a lightweight means for attaching a nasal tube to the outside of the patient's nose, while maintaining a secure hold on the tube. This device must be comfortable when worn for many days and flexible to allow the tube to be moved during continued care. Traditionally doctors will try and wrap tape around the patient's nose and even attach the tube to the upper lip. This can be very uncomfortable, cause undue irritation to the upper lip and be painful when removed if the hair of a male patient has become stuck to the adhesive tape. The instant invention will allow patients with facial hair to not have to be shaved in order to provide secure nasogastric tube attachment.

### BRIEF DESCRIPTION OF THE DRAWING

FIG. 1 shows the basic style of the flexible adhesive strip which is the basis of the Fastener. One side of the Fastener has a pressure sensitive adhesive (5) which is designed to stick to the skin and securely hold the nasogastric tube. The flaps (3 & 4) protrude from the central strip (2) and are provided for wrapping around the tube and forming a strong, secure yet flexible bond with the nasogastric tube. The central strip is also attached to the tube by means of the PSA and provides an even greater surface area for contacting and holding the tube. The large flap at the top of the device (1) is to be attached to the side and bridge of the patient's nose.

### DETAILED DESCRIPTION OF THE INVENTION

The present invention relates to a device for attaching a nasogastric tube to the patient's nose. For the purpose of this application, a nasogastric tube is any tube that is inserted through the nasal canal into the stomach. The material from which the device is made can be any type of fabric or cloth. It can also be prepared from a plastic or synthetic material or film. Non-limiting examples of materials are cotton, rayon, nylon, polyethylene, denim, silk or any high elastic compress cohesive bandage having a combination of a rubber reinforced woven yarns and a latex coating which allows cohesive properties. Examples of such products are Lycra® or Coband® (3M Center, St. Paul, Minn.). In one embodiment the fastener is made out of a high elastic rubber reinforced woven fabric.

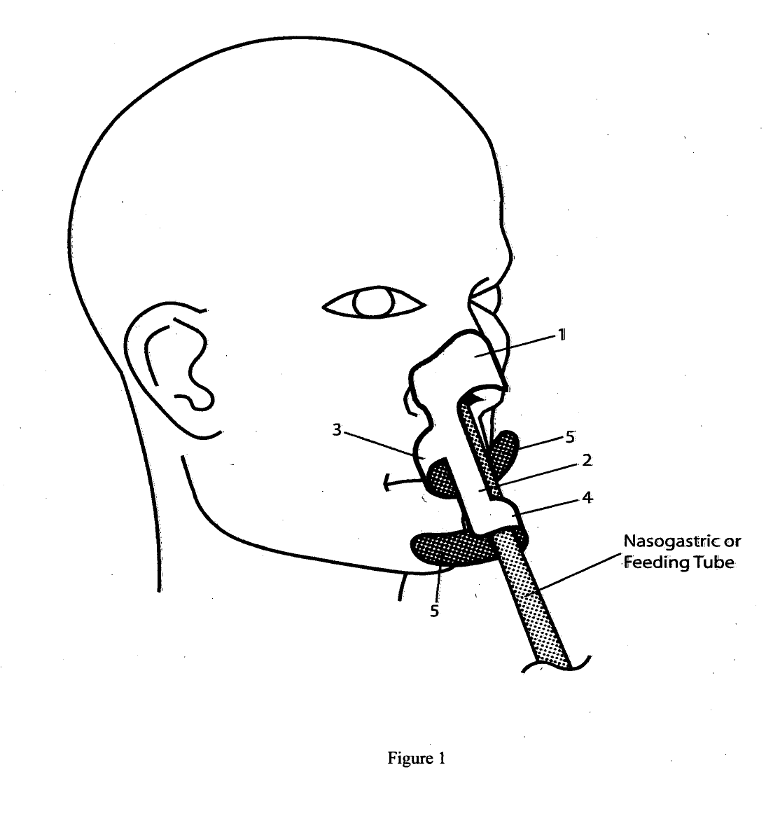

The device is attached to the nose by use of a pressure sensitive adhesive (PSA) which is applied to one side of the device (FIG. 1, #5). While virtually any PSA will function with the instant invention, one skilled in the art will recognize that issues such as skin irritation, allergic reaction, and proper adhesion to moist or oily skin all need to be considered in the choice of adhesive. Since the device must also attach to the plastic or rubber tube, the choice of adhesive is paramount. While, for sake of comfort the fabric must be flexible and provide many degrees of movement. The PSA must not significantly inhibit the flexibility of the fabric and while being worn or holding the tube. As an added benefit, the PSA should firmly attached to the nasogastric tube, while still being easily removable from the tube so that it may be repositioned. A small section at the end of flats 3 and 4 may be intentionally not coated with the PSA so as to be a point to grab the flats and remove them from the tube. Many times on insertion of the nasograstric tube, the doctor or nurse will find that the tube has not been fully inserted and has not reached the stomach. In this case, the fastener must be undone from the tube and and further inserted. It is important that the portion that was originally taped to the fastener be completely free of adhesive and pieces of the fastener, as these will cause discomfort and irritation to the patient as the tube is inserted further into the nose.

The device is applied to the nose of the patient or wearer by means of the large flap at the upper end of the device (1), and to the tube by means of the smaller flaps 3, and 4. Additionally the tube is attached along the length of the central strip (2) which provides a very high level of strength and connectivity.

In order to provide a good adhesion to the surface of the nose, it is highly desirable to have the oil and sweat removed. This can be accomplished with isopropanol (alcohol) and a swab. An optional skin barrier may be applied at this point. The. purpose of the skin barrier is to reduce irritation and promote adhesion. There are a number of commercial preparations for skin barrier, two such examples are Cavilon® Barrier film by 3M (3M Center, St. Paul, Minn.) or Brad Barrier Film® by Smith & Nephew (Healthcare House, Goulton Street, Hull, UK)

The device can be created in a variety of sizes to accommodate the wide variety of human noses. Also the flaps can be positioned as shown in FIG. 1 or in a mirrored image so that the device can be used on the other nostril. In one embodiment flap 1 will be between 3.0 and 6.0 cm wide and 3.0 to 5.0 cm long. In another embodiment the central strip will be between 3 and 6.0 cm long and 0.5 to 1.5 cm wide. The device will have at least 2 flaps (3 & 4) but depending on the strength of bond needed between the tube and hold may have more than two flaps. In on embodiment the device will have two flaps for holding the tube. Each of the flaps for holding the tube will be between 0.5 and 1.5 cm in width and 2.0 to 4.0 cm long for wrapping around the tube.

The device will cut to form the shape as illustrated in FIG. 1. The PSA is applied to one side of a sheet (large enough to allow he device to be made in one piece). The PSA is applied to one side of a sheet of material, chosen from the list above. The PSA can be applied from a solvent in the liquid form or as a hot melt in which the PSA is heated to temperature to soften the PSA to allow spreading a thin film on the one side of the device. Once the adhesive has been applied to the device, a release film or backing is applied to the adhesive side of the device to protect the adhesive from dirt and other foreign matter. The sheet is then cut, by typical means such as sissors or die cutting machine, to the desired configuration of the device. Any method for cutting the fabric and release backing cleanly without deforming the device may be used. Examples of release backings are silicon coated paper and polyethylene.

In addition to supporting the tube and fastening it to the patient's nose, the fabric should also be permeable to moisture such as sweat.

Films and fabrics that are not permeable with trap the patients sweat and will quickly loose adhesion to the skin and become ineffective. Most fabrics will provide this function if the PSA is applied in a manor such as not to create a continuous film across the surface of the fabric.

Included in this application is a method for inserting and attaching the nasogastric tube to a patient. The method consists of lubricating the tube, inserting the tube into the nose and continuing into the stomach, cleaning the lubricant from the tube, cleaning the oil and sweat for the nose, attaching the fastener to the bridge of the patients nose and then attaching the fastener to the tube first along the shaft and then by wrapping the flaps around the tube.

What is claimed:

1. A nasogastric tube Fastener comprising a fabric with a top side and a bottom side; the fabric having the shape of a top flap for attaching to a nose of a patient, a central strip that protrudes perpendicular from an edge of one end of the top flap; a plurality of bottom flaps that protruding perpendicular from alternate edges of the central strip for attaching to a nasogastric tube, and a pressure sensitive adhesive applied to the bottom side of the fabric.

2. The tube Fastener of claim 1, wherein the top flap is between 3.0 and 6.0 cm wide and 3.0 to 5.0 cm long.

3. The tube fastener of claim 1 wherein the plurality of bottom flaps are two.

4. The tube fastener of claim 1 wherein the plurality of bottom flaps are easily repositionable.

5. The tube fastener of claim 1 wherein the fabric is permeable to sweat and moisture.

6. The tube fastener of claim 5 wherein the fabric is a high elastic rubber reinforced woven yarn.

7. The tube fastener of claim 5 wherein the fabric is nylon or cotton.

8. A method of holding a nasogastric tube using the tube Fastener of claim 1.

9. A process for making a nastogastric tube fastener consisting of the steps;
lubricating a nasogastric tube;
inserting the tube in a patients nose;
cleaning the lubricant from the tube;
cleaning the patients nose to remove oil and sweat;
placing a nasogastric tube fastener to the clean nose;
attaching a center strip of the fastener to the tube, and
wrapping a plurality of flaps around the tube.

10. The process of claim 9 wherein an optional step of applying a skin barrier prior to attaching the fastener to the nose.

11. A process for making a nasogastric tube fastener consisting of;
treating a first side of a piece of fabric with a pressure sensitive adhesive (PSA);
placing a piece of release material over the pressure sensitive adhesive;
cutting the fabric and release material to a shape as shown in FIG. 1.

12. The process of claim 11 wherein the PSA is applied from a solvent.

13. The process of claim 11 wherein the PSA is applied as a hot melt.
